(19) United States
(12) Patent Application Publication
(10) Pub. No.: US 2022/0096503 A1
(43) Pub. Date: Mar. 31, 2022

(54) COMBINATION PHARMACEUTICAL COMPOSITIONS AND METHODS THEREOF

(71) Applicant: University of Washington, Seattle, WA (US)

(72) Inventors: Rodney J.Y. Ho, Seattle, WA (US); Jesse Yu, Seattle, WA (US); Lisa McConnachie, Seattle, WA (US)

(73) Assignee: University of Washington, Seattle, WA (US)

(21) Appl. No.: 17/422,074

(22) PCT Filed: Jan. 10, 2020
(86) PCT No.: PCT/US2020/013170

§ 371 Date: Jul. 9, 2021

Related U.S. Application Data
(60) Provisional application No. 62/791,453, filed on Jan. 11, 2019.

(51) Int. Cl. A61K 31/675; A61K 47/24; A61K 9/00; A61K 31/506; A61K 31/513; A61K 31/5365; A61K 31/427; A61K 31/505; A61K 9/16

(52) U.S. Cl. CPC A61K 31/675; A61K 47/24; A61K 9/0019; A61K 31/506; A61K 9/1694; A61K 31/5365; A61K 31/427; A61K 31/505; A61K 31/513

(57) ABSTRACT

Described herein are combination pharmaceutical compositions including a combination of hydrophilic and hydrophobic therapeutic agents (i.e., drugs) that are assembled together with excipients under specific conditions, forming a homogeneous pharmaceutical powder with unified repetitive multi-drug motif (MDM) structure. Unlike currently available drug combination powders, which are amorphous, the combination pharmaceutical compositions (e.g., combination therapeutic agent powders) of the present disclosure have long range order, in the form of repetitive multi-drug and unified motifs

### CROSS-REFERENCE(S) TO RELATED APPLICATION(S)

This application claims the benefit of U.S. Patent Application No. 62/791,453, filed Jan. 11, 2019, the disclosure of which is incorporated herein by reference in its entirety.

### STATEMENT OF GOVERNMENT LICENSE RIGHTS

This invention was made with government support under Grant No. UM1 AI120176, awarded by the National Institutes of Health. The government has certain rights in the invention.

### BACKGROUND

The availability of well-characterized viral protein structures coupled with success in developing viral protein inhibitors has significantly improved human immunodeficiency virus (HIV) treatment over the years. To address the rapid evolution of HIV and HIV drug resistance, oral drug combinations are often used to target multiple HIV proteins or different binding sites on the same protein. This therapeutic approach is the current standard of care in HIV and is referred to as oral combination antiretroviral treatment (cART) or highly active antiretroviral treatment (HAART). Orally administered cART or HAART with two or three drug combinations target HIV at multiple checkpoints in replication. In doing so, the cART approach has been successful in suppressing the HIV virus to undetectable levels in plasma and has reduced the risk of harboring drug resistance. While these treatment regimens have significantly reduced the mortality of HIV-infected patients, these chronic oral regimens are associated with significant pill burden and require diligent patient adherence to daily oral dosing.

Fixed dose oral combination products have been used to reduce pill burden and improve patient compliance. Due to the diverse morphology and rheology of the individual drug constituents in fixed dose combinations, powder uniformity is an important parameter for drug product development. For orally administered cART, heterogeneous distribution of the active pharmaceutical ingredients (API) can be particularly problematic. The physical properties of drug substances or API used in combination HIV treatment can vary widely and can range from very hydrophilic (e.g., nucleoside reverse transcriptase inhibitors [NRTI]) to very hydrophobic (e.g., integrase strand transfer inhibitors [INSTI] and protease inhibitors [PI]). As a result, fixed dose combinations can experience variable dissolution profiles in vivo if hydrophobic- or hydrophilic-rich domains are present.

The complex interactions of APIs and excipients in the solid state can impact the stability and bioperformance of pharmaceutical products. Interaction of drugs with excipients can be facilitated through a number of processes including milling, lyophilization, hot melt extrusion, and solvent evaporation. Traditionally, spray drying is used to combine drugs and excipients for pharmaceutical products by atomizing liquid feedstock into a heated inert gas to rapidly remove a solvent under uncontrolled conditions, thereby providing dried amorphous particles, which can increase the aqueous solubility of hydrophobic biomolecules. However, while the increase in aqueous solubility can be desirable, the amorphous materials are thermodynamically unstable and spontaneously or readily revert to more stable structures in a process known as devitrification.

Thus, there is a need for combinations of therapeutic agents that are stable, and that can provide combination pharmaceutical formulations that can extend the plasma drug concentrations of each therapeutic agent component over periods of days and/or weeks. The present disclosure fulfils these needs and provides further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a method of making a combination pharmaceutical composition, including dissolving a hydrophobic therapeutic agent having a log P value of 1 or greater; a hydrophilic therapeutic agent having a log P value of less than 1; and one or more compatibilizers comprising a lipid excipient, a lipid conjugate excipient, or a combination thereof in an alcoholic solvent at a temperature of 65 to 75° C. to provide a solution, maintaining the solution at a temperature of 65 to 75° C.; spraying the solution from an inlet nozzle and evaporating the alcoholic solvent in a chamber to provide the combination pharmaceutical composition in the form of a powder, including the hydrophobic therapeutic agent, the hydrophilic therapeutic agent, and the one or more compatibilizers.

In another aspect, the present disclosure features a combination pharmaceutical composition made according to the methods described herein. The combination pharmaceutical composition includes a hydrophobic therapeutic agent having a log P value of 1 or greater; a hydrophilic therapeutic agent having a log P value of less than 1; and one or more compatibilizers comprising a lipid excipient, a lipid conjugate excipient, or a combination thereof. The combination pharmaceutical composition has a powder X-ray diffraction pattern that includes at least one peak having a signal to noise ratio of greater than 3, wherein the peak is different from the diffraction peaks of each individual component of the combination pharmaceutical composition.

In another aspect, the present disclosure features a method of administering the combination pharmaceutical compositions described herein, including mixing a combination pharmaceutical composition with an aqueous solvent to provide an aqueous dispersion including the combination pharmaceutical composition; and parenterally administering the aqueous dispersion to a subject.

In yet a further aspect, the present disclosure features a suspension including a combination pharmaceutical composition described herein, dispersed in an aqueous solvent in the form of a suspension.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1A shows the powder X-ray diffraction pattern of lopinavir (LPV).

FIG. 1B shows the powder X-ray diffraction pattern of ritonavir (RTV).

FIG. 1C shows the powder X-ray diffraction pattern of tenofovir (TFV).

FIG. 1D shows the powder X-ray diffraction pattern of DSPC.

FIG. 1E shows the powder X-ray diffraction pattern of DSPE-PEG₂₀₀₀.

FIG. 1F shows the powder X-ray diffraction pattern of physically mixed LPV/RTV/TFV/DSPC/DSPE-PEG₂₀₀₀. The constituents of the quinternary mixture show sharp diffraction peaks unique to their crystal lattices. The diffraction pattern of the physical mixture has characteristics of DSPC due to the high mass % of the DSPC but also has additional peaks from the other components.

FIG. 1G shows the powder X-ray diffraction pattern of spray-dried DSPC/DSPE-PEG₂₀₀₀.

FIG. 1H shows the powder X-ray diffraction pattern of an embodiment of a pharmaceutical composition of the present disclosure. The pharmaceutical composition has spray-dried LPV/RTV/TFV/DSPC/DSPE-PEG₂₀₀₀. The composition has two distinct peaks indicative of new long range order generated by the spray drying process. The loss of peaks in 19.1° 2θ and 23.1° 2θ attributable to the PEG moiety of the spray-dried lipid and lipid conjugate excipients after addition of drugs indicated that drug-PEG interactions can prevent crystallization of PEG.

FIG. 2 shows the differential scanning calorimetry (DSC) patterns of combination antiretroviral drugs and excipients. The constituents of the physical mixture show unique endothermic transitions. The physical mixture of all 5 constituents shows a complex thermogram with multiple endothermic transitions. Line a is the DSC trace of tenofovir, line b is the DSC trace of lopinavir, line c is the DSC trace of ritonavir, line d is the DSC trace of DSPC, line e is the DSC trace of DSPE-PEG₂₀₀₀, line f is the DSC trace of physically mixed LPV/RTV/TFV/DSPC/DSPE-PEG₂₀₀₀, line g is the DSC trace of an embodiment of a pharmaceutical composition of the present disclosure, specifically a spray-dried composition of LPV/RTV/TFV/DSPC/DSPE-PEG₂₀₀₀, the spray-dried combination powder has a single endothermic transition observed at 74.29° C. This melting point was not attributable to any of the other melting points seen in FIG. 2, line h is the DSC trace of spray-dried DSPC/DSPE-PEG₂₀₀₀. The lipid and lipid conjugate excipient powder shows multiple endotherms, indicating that the presence of therapeutic agents can prevent crystallinity in these powders in corroboration with the powder X-ray diffraction data in FIGS. 1A-1G.

FIGS. 3A and 3B show scanning electron micrographs of morphological changes associated with the spray-drying process.

FIG. 3A is a scanning electron micrograph of a physically mixed composition including therapeutic agents and excipients.

FIG. 3B is a scanning electron micrograph of an embodiment of a pharmaceutical composition of the present disclosure, made by spray drying. The micrograph shows that after spray-drying, a significant shift occurred toward spherical geometries. In addition, a subset of the spherical particles had local cavitation and wrinkling present on their surfaces.

FIGS. 4A-4F show the ToF-SIM (Time of Flight Secondary Ion Mass Spectrometry)_analysis of a homogeneous distribution of therapeutic agents and excipients in an embodiment of a pharmaceutical composition of the present disclosure relative to the physically-mixed controls.

FIG. 4A is a ToF-SIM analysis of ritonavir (red, mass fragment of 59 AMU), lopinavir (green, mass fragment of 101.07 AMU), and tenofovir (blue, mass fragment of 148.04). The figure generated is a composite of X, Y and Z axis, with Z-planes overlaid on top of each other.

FIG. 4B is a is a ToF-SIM analysis of DSPC (red, mass fragment of 58.02) and DSPE-PEG₂₀₀₀ (green, mass fragment of 61.03). The figure generated is a composite of X, Y and Z axis, with Z-planes overlaid on top of each other.

FIG. 4C is a pixel analysis using ImageJ software to show the relative abundance of pixels over the X-coordinate of each image. Within the micron scale, both therapeutic agent and excipients were homogeneously dispersed with no concentrated drug or excipient domains in the spray-dried material (FIGURES A, B and C).

FIG. 4D is a ToF-SIM analysis of ritonavir (red, mass fragment of 59 AMU), lopinavir (green, mass fragment of 101.07 AMU), and tenofovir (blue, mass fragment of 148.04). The figure generated is a composite of X, Y and Z axis, with Z-planes overlaid on top of each other.

FIG. 4E is a is a ToF-SIM analysis of DSPC (red, mass fragment of 58.02) and DSPE-PEG₂₀₀₀ (green, mass fragment of 61.03). The figure generated is a composite of X, Y and Z axis, with Z-planes overlaid on top of each other.

FIG. 4F is a pixel analysis using ImageJ software to show the relative abundance of pixels over the X-coordinate of each image. Within the micron scale, there were concentrated regions of drugs and excipients in the physically-mixed controls (FIGURES D, E and F).

FIG. 5A is a graph comparing the x-ray diffraction pattern of an embodiment of a pharmaceutical composition of the present disclosure.

FIG. 5B is a graph comparing the x-ray diffraction pattern of a lyophilized composition.

FIG. 6 is a flow chart showing a process of making an aqueous suspension of an embodiment of a pharmaceutical composition of the present disclosure.

FIG. 7A shows the scanning electron micrograph of an embodiment of a pharmaceutical composition of the present disclosure in suspension in an aqueous medium. The embodiment of the pharmaceutical composition of the present disclosure forms nanoparticles in suspension, without formation of a bilayer structure.

FIG. 7B shows the scanning electron micrograph of a comparative liposome composition in suspension in a liquid medium.

FIG. 8 is an X-ray diffraction pattern showing a multi-drug motif (MDM) structure found in an embodiment of a pharmaceutical composition of the present disclosure compared to amorphous forms of individual therapeutic agent components (lopinavir and ritonavir).

### DETAILED DESCRIPTION

The present disclosure provides combination pharmaceutical compositions including a combination of hydrophilic and hydrophobic therapeutic agents that are assembled together with excipients under specific conditions, forming a homogeneous pharmaceutical powder with a unified repetitive multi-drug motif (MDM) structure (used interchangeably herein with “multi-drug-lipid motif” and “multi-drug motif”). Unlike currently available drug combination powders, which are amorphous, the combination pharmaceutical compositions (e.g., combination therapeutic agent powders) of the present disclosure have long range order, in the form of repetitive multi-drug and unified motifs.

The combination pharmaceutical compositions are made by fully dissolving all therapeutic agents and excipients in an alcoholic solvent, which can optionally include water or a water-based buffer; followed by a controlled solvent removal process that locks the therapeutic agent and excipients into multi-drug motifs (MDM) with long range translational periodicity. These motifs are structurally different from purely amorphous material as verified by powder x-ray diffraction, and the combination pharmaceutical composition can be hydrated and homogenized to produce a long-acting injectable suspension with both hydrophilic and hydrophobic therapeutic agents, having stable release profiles. The process of controlled solvent removal from the solution of therapeutic agents and excipients is important to generate a combination pharmaceutical composition with MDM. The resulting combination pharmaceutical composition is stable, and can provide long-acting therapeutic combinations having extended plasma therapeutic agent concentrations for the therapeutic agent components, compared to separately administered individual therapeutic agent components, or an amorphous mixture of the therapeutic agents and excipients.

### Definitions

At various places in the present specification, groups or ranges are described. It is specifically intended that the disclosure include each and every individual subcombination of the members of such groups and ranges.

The verb “comprise” and its conjugations, are used in the open and non-limiting sense to mean that items following the word are included, but items not specifically mentioned are not excluded.

“About” in reference to a numerical value refers to the range of values somewhat less or greater than the stated value, as understood by one of skill in the art. For example, the term “about” could mean a value ranging from plus or minus a percentage (e.g., ±1%, ±2%, or ±5%) of the stated value. Furthermore, since all numbers, values, and expressions referring to quantities used herein are subject to the various uncertainties of measurement encountered in the art, then unless otherwise indicated, all presented values may be understood as modified by the term “about.”

As used herein, the articles “a,” “an,” and “the” may include plural referents unless otherwise expressly limited to one-referent, or if it would be obvious to a skilled artisan from the context of the sentence that the article referred to a singular referent.

Where a numerical range is disclosed herein, then such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of 1 and the maximum value of 10. Exemplary subranges of the range “1 to 10” include, but are not limited to, e.g., 1 to 6.1, 3.5 to 7.8, and 5.5 to 10.

As used herein, the term “matrix” denotes a solid mixture composed of a continuous phase, and one or more dispersed phase(s) (e.g., particles of the pharmaceutically active agent).

The terms “therapeutic agent”, “active agent”, “drug”, and “active pharmaceutical ingredient” are used interchangeably herein.

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety or a molecule that is not attracted to water with significant apolar surface area at physiological pH and/or salt conditions. This phase separation can be observed via a combination of dynamic light scattering and aqueous NMR measurements. A hydrophobic therapeutic agent has a log P value of 1 or greater.

As used herein, the term “hydrophilic” refers to a moiety or a molecule that is attracted to and tends to be dissolved by water. The hydrophilic moiety is miscible with an aqueous phase. A hydrophilic therapeutic agent has a log P value of less than 1.

The log P values of hydrophobic and hydrophilic drugs can be found, for example, at pubchem.ncbi.nlm nih.gov and drugbank.ca.

As used herein, the log P value is a constant defined in the following manner:

Log P=log 10(Partition Coefficient)

Partition Coefficient, P=[organic]/[aqueous]

where [ ] indicates the concentration of solute in the organic and aqueous partition. A negative value for log P means the compound has a higher affinity for the aqueous phase (it is more hydrophilic); when log P=0 the compound is equally partitioned between the lipid and aqueous phases; a positive value for log P denotes a higher concentration in the lipid phase (i.e., the compound is more lipophilic). Log P=1 means there is a 10:1 partitioning in organic: aqueous phases. The most commonly used lipid and aqueous system is octan-1-ol and water, or octanol and buffer at a pH of 6.5 to 8.5.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “polymer” refers to a macromolecule having more than 10 repeating units.

As used herein, the term “small molecule” refers to a low molecular weight (<2000 daltons) organic compound that may help regulate a biological process, with a size on the order of 1 nm. Most drugs are small molecules.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Combination Pharmaceutical Compositions

The present disclosure features, inter alia, a combination pharmaceutical composition, including one or more hydrophobic therapeutic agents having a log P value of 1 or greater; one or more hydrophilic therapeutic agents having a log P value of less than 1; and one or more compatibilizers such as a lipid excipient, a lipid conjugate excipient, or a combination thereof. The combination pharmaceutical composition has a powder X-ray diffraction pattern that has at least one peak having a signal to noise ratio of greater than 3 (e.g., greater than 4, greater than 5, or greater than 6). The at least one peak has a different 2θ peak position than the diffraction peak 2θ positions of each individual component (e.g., each individual therapeutic agent, or each individual therapeutic agent and excipient) of the combination pharmaceutical composition. The at least one peak has a different 2θ peak position than the diffraction peak 2θ positions for a simple physical mixture of the individual components of the combination pharmaceutical composition. The X-ray diffraction pattern of the combination pharmaceutical composition is indicative of multiple therapeutic agents assembled into a unified domain having repeating identical units, such that the hydrophobic therapeutic agent, the hydrophilic agent, and the one or more compatibilizers together form an organized composition. The composition can have a long range order in the form of a repeating pattern. As used herein, short range order involves length scales of from 1 Å (or 0.1 nm) to 10 Å (or 1 nm), while long range order has length scales that exceed 10 nm, or of an order that is at 2 theta 10-25 nm. Thus, the combination pharmaceutical composition of the present disclosure has a unified repetitive multi-drug motif (MDM) structure and is referred to interchangeably herein as an “MDM composition”.

In some embodiments, the combination pharmaceutical composition remains stable when stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months), such that the at least one X-ray diffraction peak at position(s) corresponding to the combination pharmaceutical composition are preserved over the time period. In some embodiments, both the X-ray diffraction peak positions and intensities are preserved when the composition is stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months).

The combination pharmaceutical composition of the present disclosure is not amorphous, and is not an amorphous solid dispersion. The combination pharmaceutical composition is not a physical mixture or blend of its constituent therapeutic agents and excipients, and as such, possess properties unique to the composition that are different from those of each of the constituent therapeutic agents and excipients. For example, the combination pharmaceutical composition can have a phase transition temperature different from the transition temperature of each individual component when assessed by differential scanning calorimetry. In some embodiments, one or more of the transition temperatures of each individual component is no longer present in the combination pharmaceutical composition, which includes an organized assembly of the therapeutic agent and excipient components. In some embodiments, the combination pharmaceutical composition has a homogeneous distribution of each individual therapeutic agent when viewed by scanning electron microscopy, such that each individual component is not visually discernible at 10-20 kV.

In some embodiments, the hydrophobic therapeutic agent(s) and the hydrophilic therapeutic agent(s) contained in the combination pharmaceutical composition are each a small molecule having a molecular weight of less than 2000 (e.g., less than 1500, less than 1000, less than 500, or from 300 to 1000). In some embodiments, the combination pharmaceutical composition can include one or more hydrophobic therapeutic agents in an amount of 2 wt % or more (e.g., 5 wt % or more, 10 wt % or more, or 15 wt % or more) and/or 20 wt % or less (e.g., 15 wt % or less, 10 wt % or less, or 5 wt % or less) relative to the weight of the total combination pharmaceutical composition. The hydrophobic therapeutic agent can include a hydrophobic antiviral agent and/or a hydrophobic anti-infective agent (e.g., a hydrophobic antimicrobial agent such as amphotericin). For example, the hydrophobic antiviral agent can be lopinavir, ritonavir, dolutegravir, rilpivirine, atazanavir, dorunavir, efevirenz, and/or raltigravir.

In some embodiments, the composition includes one or more hydrophilic therapeutic agents in an amount of 2 wt % or more (e.g., 5 wt % or more, 10 wt % or more, or 15 wt % or more) and/or 20 wt % or less (e.g., 15 wt % or less, 10 wt % or less, or 5 wt % or less) relative to the weight of the total combination pharmaceutical composition. The hydrophilic agent can include an antiviral agent and/or an anti-infective agent (e.g., a hydrophilic antimicrobial agent such as vancomycin). For example, the hydrophilic antiviral agent can include lamivudine, abacavir, tenofovir and its prodrugs (e.g., tenofovir disoproxil fumarate, tenofovir alafenamide), and emtricitabine.

The combination pharmaceutical composition can include the one or more compatibilizers in an amount of 60 wt % or more (e.g., 70 wt % or more, 80 wt % or more, 90 wt % or more) and 95 wt % or less (e.g., 90 wt % or less, 80 wt % or less, or 70 wt % or less) relative to the weight of the total combination pharmaceutical composition. The one or more compatibilizers can include at least one lipid excipient and at least one lipid conjugate excipient. For example, the one or more compatibilizers can include at least one lipid excipient in an amount of 50 wt % or more and 80 wt % or less. The lipid excipient can be a saturated or unsaturated lipid excipient, such as a phospholipid. The phospholipid can include, for example, 1,2-distearoyl-sn-glycero-3-phosphocholine (DSPC), 1,2-dimyristoyl-sn-glycero-3-phosphocholine (DMPC), 1,2-dipalmitoyl-sn-glycero-3-phosphocholine (DPPC). In some embodiments, the one or more compatibilizers include at least one lipid conjugate excipient in an amount of 19 wt % or more and 25 wt % or less relative to the weight of the total combination pharmaceutical composition. The lipid conjugate excipient can be a covalent conjugate of a lipid with a hydrophilic moiety. The hydrophilic moiety can include a hydrophilic polymer, such as poly(ethylene glycol) having a molecular weight (Mₙ) of from 500 to 5000 (e.g., from 500 to 4000, from 500 to 3000, from 500 to 2000, from 1000 to 5000, from 1000 to 4000, from 1000 to 3000, from 1000 to 2000, from 2000 to 5000, from 2000 to 4000, from 2000 to 3000, 2000, 1000, 5000, or 500). In some embodiments, the lipid conjugate excipient is a conjugate of 1,2-distearoyl-sn-glycero-3-phosphoethanolamine (DSPE) with PEG, such as PEG₂₀₀₀. The PEG can be conjugated to the lipid via an amide linkage. The lipid conjugate excipient can be in the form of a salt, such as an ammonium or a sodium salt.

The combination pharmaceutical composition can include a molar ratio of the sum of hydrophobic therapeutic agent and hydrophilic therapeutic agent, to the one or more compatibilizers, of from 30:115 to 71:40 (e.g., from 40:115 to 71:40, from 50:100 to 71:40, from 60:100 to 71:40, from 70:100 to 71:40, from 70:90 to 71:50, from 70:80 to 71:50, or from 70:70 to 71:50).

The combination pharmaceutical composition can be a solid. For example, the combination pharmaceutical composition can be a powder. The powder can be formed of particles having an average dimension of from 100 nm (e.g., from 500 nm, from 1 μm, from 4 μm, from 6 μm, or from 8 μm) to 10 μm (e.g., to 8 μm, to 6 μm, to 4 μm, to 1 μm, or to 500 nm). The average dimension (e.g., a diameter) of a particle can be determined by transmission and/or scanning electron microscopy.

### Administration

The combination pharmaceutical composition of the present disclosure are suitable for parenteral administration, when suspended in an aqueous solvent. Thus, the present disclosure features, inter alia, a method of administering the combination pharmaceutical composition described above, including mixing the combination pharmaceutical composition with an aqueous solvent to provide an aqueous dispersion. The aqueous dispersion can be a suspension of the combination pharmaceutical composition, which can initially be in the form of a powder. In some embodiments, once suspended in the aqueous solvent, the size of the suspended particles of the combination pharmaceutical composition is reduced (e.g., to less than 0.2 μm), for example, by subjecting the aqueous dispersion to a homogenizer and/or a sonicator. The aqueous dispersion can then be optionally filtered to remove any microorganisms, for example, through a 0.2 μm filter. The aqueous dispersion is adapted to be parenterally administered to a subject. As used herein, parenteral administration refers to a medicine taken into the body or administered in a manner other than through the digestive tract, such as by intravenous administration or intramuscular injection.

The particles of combination pharmaceutical composition in the aqueous dispersion can maintain the supramolecular MDM organization of the hydrophobic therapeutic agent, the hydrophilic therapeutic agent, and the one or more compatibilizer. In some embodiments, the particles of the combination pharmaceutical composition in the aqueous dispersion do not form a lipid layer, a lipid bilayer, a liposome, or a micelle in the aqueous solvent. In some embodiments, after hydration of the combination pharmaceutical composition, the particles of combination pharmaceutical composition are discoidal rather than spherical, when visualized by transmission electron microscopy. For example, the discoid particles of the combination pharmaceutical composition can have a dimension of, for example, a width of from 5 nm (e.g., from 8 nm, from 10 nm, or from 15 nm) to 20 nm (e.g., to 15 nm, to 10 nm, or to 8 nm) by a length of from 30 nm (e.g., from 35 nm, from 40 nm, or from 45 nm) to 50 nm (e.g., to 45 nm, to 40 nm, or to 35 nm), having a thickness of from 3 nm (e.g., from 5 nm, from 7 nm) to 10 nm (e.g., to 7 nm, to 5 nm), as visualized by transmission electron microscopy.

The particles of the combination pharmaceutical composition can have a maximum dimension of from 10 nm (e.g., 25 nm, 50 nm, 100 nm, 150 nm, 200 nm) to 300 nm (e.g., 200 nm, 150 nm, 100 nm, 50 nm, or 25 nm).

In some embodiments, the aqueous solvent is a buffered aqueous solvent, saline, or any balanced isotonic physiologically compatible buffer suitable for administration to a subject, as known to a person of skill in the art. For example, the aqueous solvent can be an aqueous solution of 20 mM sodium bicarbonate and 0.45 wt % to 0.9 wt % NaCl.

In some embodiments, the aqueous dispersion includes the combination pharmaceutical composition in an amount of 10 wt % or more (e.g., 15 wt % or more, or 20 wt % or more) and 25 wt % or less (e.g., 20 wt % or less, or 15 wt % or less), relative to the final aqueous dispersion.

In certain embodiments, rather than providing a suspension of the combination pharmaceutical composition in an aqueous solvent, where the combination pharmaceutical composition is present in the form of insoluble particles suspended in the aqueous solvent, the method can include dissolving the combination pharmaceutical composition in an aqueous solvent to provide a solution. When the combination pharmaceutical composition is in a solution, it is solubilized and dissolved in the solvent.

The aqueous dispersion of the combination pharmaceutical composition of the present disclosure can provide a therapeutically effective plasma concentration of the therapeutic agents over a longer period of time compared an aqueous dispersion of a physical mixture of the therapeutic agents and excipients, an amorphous mixture of the therapeutic agents and excipients, or compared to separately administered therapeutic agents at a same dosage. In some embodiments, the aqueous dispersion of the combination pharmaceutical composition provides from 2 (e.g., from 5, from 10, or from 15) to 20 (e.g., to 15, to 10, or to 5) fold higher exposure (e.g., AUC₀₋₂₄ₕ calculated from plasma drug concentrations using the trapezoidal rule) of the therapeutic agents in non-human primates, when administered parenterally (e.g., subcutaneously), when compared to non-human primates treated with an equivalent dose of the same free and soluble therapeutic agent combination in solution. In some embodiments, the aqueous dispersion of the combination pharmaceutical composition provides from 2 fold (e.g., from 5 fold, from 10 fold, from 15 fold, from 20 fold, or from 25 fold) to 29 fold (e.g., to 25 fold, to 20 fold, to 15 fold, to 10 fold, or to 5 fold) higher exposure (e.g., AUC₀₋₂₄ₕ calculated from plasma drug concentrations using the trapezoidal rule) of the therapeutic agents in non-human primates, when administered parenterally (e.g., subcutaneously), when compared to non-human primates treated with an equivalent dose of the same free and soluble therapeutic agent combination in solution.

In some embodiments, the aqueous dispersion of the combination pharmaceutical composition of the present disclosure is long-acting, such that the parenteral administration of the aqueous dispersion can occur once per 7 (e.g., per 10, per 14, or per 18) to 28 (e.g., to 18, to 14, or to 10) days.

In certain embodiments, the aqueous dispersion of the combination pharmaceutical composition of the present disclosure has a terminal half-life greater than the terminal half-life of each freely solubilized individual therapeutic agent. For example, the combination pharmaceutical composition and aqueous dispersions thereof can have a half-life extension of greater than 2 to 3 fold of each constituent therapeutic agent's individual elimination half-life. In some embodiments, the combination pharmaceutical composition and aqueous dispersions thereof can have a half-life extension of from 8 fold (e.g., from 10 fold, from 15 fold, from 20 fold, from 30 fold, from 40 fold, or from 50 fold) to 62 fold (e.g., to 50 fold, to 40 fold, to 30 fold, to 20 fold, to 15 fold, or to 10 fold) for each constituent therapeutic agent's individual elimination half-life.

### Method of Making the Combination Pharmaceutical Composition

The combination pharmaceutical compositions of the present disclosure are made via a controlled evaporation of a solvent for solubilized therapeutic agents and excipients. In particular, the formulation method includes dissolving one or more hydrophobic therapeutic agents having a log P value of 1 or greater; one or more hydrophilic therapeutic agents having a log P value of less than 1; and one or more compatibilizers comprising a lipid excipient, a lipid conjugate excipient, or a combination thereof, in an alcoholic solvent at a temperature of 65 to 75° C. to provide a solution. The one or more hydrophobic therapeutic agents, the one or more hydrophilic therapeutic agents, and the compatibilizer(s) can be fully solubilized in the alcoholic solvent to provide a visually clear solution. The solution is maintained at a temperature of 65° C. to 75° C., and is sprayed from an inlet nozzle into a chamber, where the alcoholic solvent is evaporated in a controlled manner at a suitable temperature and pressure to provide the combination pharmaceutical composition, which includes particles of homogeneously distributed hydrophobic therapeutic agent(s), hydrophilic therapeutic agent(s), and one or more compatibilizers in an organized multi-drug motif. The combination pharmaceutical composition can be in the form of a powder.

In some embodiments, spraying the solution forms droplets of the dissolved therapeutic agents and compatibilizer(s) in the alcoholic solvent. The droplets can have a diameter of 1 μm or more (e.g., 10 μm or more, 40 μm or more, 60 μm or more, 80 μm or more, 100 μm or more, 125 μm or more) and 150 μm or less (e.g., 125 μm or less, 100 μm or less, 80 μm or less, 60 μm or less, 40 μm or less, or 10 μm or less). Evaporation of the alcoholic solvent from the droplets can occur simultaneously with spraying the solution, such that evaporation of the alcoholic solvent starts immediately upon formation of the droplets. The alcoholic solvent can evaporate from the droplets while the droplets are in suspension in the atmosphere of the chamber. The combination pharmaceutical composition in the form of a powder can form while the droplets are in suspension in the atmosphere of the chamber. The powder can be further dried under vacuum for a period of time, until, for example, all solvents have been removed.

In some embodiments, the alcoholic solvent includes methanol, ethanol, propanol, or any combination thereof. In certain embodiments, the alcoholic solvent further includes water, or an aqueous buffer. In some embodiments, the hydrophobic therapeutic agent(s) and the one or more compatibilizers are first dissolved in an alcohol to provide an alcoholic solution. The hydrophobic therapeutic agent(s) and the one or more compatibilizers can be fully solubilized in alcoholic solution, such that the alcoholic solution is visually clear upon inspection. The hydrophilic therapeutic agent(s) can be separately dissolved in an aqueous solution, such as water or an aqueous buffer. In some embodiments, a minimum amount of water or the aqueous buffer agent can be used to dissolve the hydrophilic therapeutic agent(s). The aqueous solution of hydrophilic therapeutic agent(s) can then be added to the alcoholic solution of hydrophobic therapeutic agent(s) and compatibilizer(s) can then be added to provide the visually clear solution. The dissolutions of the hydrophobic therapeutic agent(s), the hydrophilic therapeutic agent(s), and the compatibilizer(s) can occur entirely or in part at a temperature of 50° C. to 75° C. (e.g., 65° C. to 75° C.). In some embodiments, the alcohol, water, and/or the aqueous buffer can have a temperature of 50° C. (e.g., 60° C., 65° C., or 70° C.) to 75° C. (e.g., 70° C., 65° C., or 60° C.).

In some embodiments, the solution, prior to droplet formation, includes 5% wt/v to 10% wt/v, cumulatively, of the hydrophobic therapeutic agent(s), the hydrophilic therapeutic agent(s), and the one or more compatibilizers.

When spraying the solution from an inlet nozzle of an instrument, the spraying can be conducted with inlet air speed of from 0.25 m³/min (e.g., or 0.30 m³/min) to 0.35 m³/min (e.g., or 0.30 m³/min), an inlet temperature can be maintained at 65° C. (e.g., or at 70° C.) to 75° C. (e.g., or to 70° C.) to promote evaporation and to maintain the solubilized nature of the solution. The chamber into which the droplets are formed can be maintained at a pressure of from 20 mBar (e.g., or from 25 mBar) to 30 mBar (e.g., or to 25 mBar). In some embodiments, the spraying can be done with a spray-drying instrument, such as ProCepT 4-M8TriX (Zelzate, Belgium), or Buchi spray-drying instrument.

The following Examples describe combination pharmaceutical compositions. Combination pharmaceutical compositions with multi-drug motifs and suspensions thereof were prepared in Example 1 and could enhance drug levels in cells in periphery and lymph nodes in non-human primates. Example 2 describes a suspended combination pharmaceutical composition product exhibiting long-acting plasma pharmacokinetics of antiviral drugs. Example 3 describes a suspended combination pharmaceutical composition that can extend antiviral plasma circulation. Example 4 is a comparison of conventional dosage form of LPV/RTV taken orally in humans compared to orally in primates. Example 5 is a comparison of conventional dosage of TFV given intravenously (IV) in humans compared to subcutaneously (SC) in primates.

### EXAMPLES

### Example 1. Generation and Characterization of Combination Pharmaceutical Compositions Having Multi-Drug Motifs

Combination multiple-drug particles were generated, having a stable drug-combination motif in a powder form. These particles were then made into a nanosuspension dosage form. The powders were not amorphous. The production of a stable and reproducible multi-drug-lipid motif (MDM) in the solid state requires a special process and composition. It is believed that the controlled removal of solvent from solubilized drugs and excipients enable generation of these multi drug motifs. Therefore, the formation, structural features and molecular distribution of multi drug motif (MDM) formulation were studied.

A drug combination in MDM motif powder form was suspended in aqueous solvent and after size-reduction, the suspended MDM composition produces a long-acting plasma, targeted effect to peripheral blood mononuclear cells in non-human primates.

Materials

GMP quality lopinavir (LPV), ritonavir (RTV) and tenofovir (TFV) were supplied by Mylan pharmaceuticals (Morgantown, W. Va.). GMP quality lipid and lipid conjugate excipients 1,2-distearoyl-sn-glycero-3-phosphocholine (DSPC) and 1,2-distearoyl-sn-glycero-3-phosphoethanolamine-N-[amino(polyethylene glycol)-2000] (DSPE-PEG2000) were purchased from Cordon Pharma (Liestal, Switzerland). Anhydrous ethanol (200 proof) was purchased from Decon Pharmaceuticals (King of Prussia, Pa.). All other reagents were of analytical grade or higher quality.

To prepare a combination pharmaceutical composition powder, all the drugs were first solubilized fully in ethanol with a small amount of aqueous buffer. Complete dissolution of all constituents was verified visually. The fully solubilized drug and excipients were subjected to controlled solvent removal through spray-drying; or a less-well controlled removal by rotary evaporation for comparison as described below. In the event that precipitation or phase separation is observed prior to solvent removal, MDM formation would be incomplete and could result in drug product failure.

13.12 g of LPV and 3.76 g of RTV were solubilized together in 70° C. ethanol with 14.22 g of DSPC and 56.11 g of mPEG2000-DSPE; 7.49 g of TFV was solubilized in 12.5 ml of 200 mM NaHCO₃ buffer and both solutions were added together to form a mixed solvent with all the three APIs and lipid and lipid conjugate excipients in solution at a temperature maintained at 75° C. The final API and excipient total concentration was kept at either 5 or 10% w/v. Solvent removal was performed with a ProCepT 4-M8TriX spray drying system (Zelzate, Belgium). Inlet temperature for the spray dryer was maintained at 70° C. with an inlet air speed of 0.3 m³/min and chamber pressure of 25 mBar. Dried powder generated by the spray-dryer was collected and subjected to vacuum desiccation for 48 hr. The dried drug-combination powder products were characterized with powder X-ray diffraction, DSC or ToF-SIM and other physical analyses described below. Control products with or without excipients were also generated either through spray drying or rotary evaporation.

To generate a suspension of the combination pharmaceutical composition, the powder was added to 0.45% w/v NaCl plus 20 mM NaHCO₃ buffer at 70° C. to achieve a nominal concentration of 10.7 mg/mL lopinavir, 3.1 mg/mL ritonavir, 6.1 mg/mL tenofovir. The suspension had a total lipid concentration of 180 mM composed of 9:1 mole to mole DSPC to DSPE-PEG2000. The suspension, after holding at 70° C. for 4 hours was subjected to size-reduction with a homogenizer (Avestin, Canada) to generate the combination pharmaceutical composition in the form of drug combination nanoparticles, in suspension.

Powder X-ray Diffraction

Powder X-ray Diffraction (PXRD) was performed on a Bruker D8 Focus X-ray Diffractor (Madison, Wis., USA) with Cu-Kα radiation. Operational voltage and amperage were set to 40.0 kV and 40.0 mA, respectively. Parameters includes a step size of 0.035° 2θ in an operating range of 5° to 50° 2θ. Powder (˜100-200 mg) was pressed into a sample container to obtain a flat upper surface.

Differential Scanning calorimetry Analysis

Differential scanning calorimetry (DSC) was performed on the dry powder of the combination pharmaceutical composition with a TA DSC Q20 (New Castle, Del., USA). Under constant nitrogen (50 mL/min), baseline calibrations were performed every day prior to instrument use by ramping 10° C./min up to 200° C. 1-4 mg of each test sample was placed in a hermetically sealed aluminum pan and samples were scanned at 10° C./min from ambient room temperature.

Scanning Electron Microscopy (SEM)

A dry powder of the combination pharmaceutical composition was visualized using a FEI Sirion XL30 Scanning Electron Microscope (Hillsboro, Oreg.). Samples were placed on a conductive and adhesive carbon backplate and placed under a nitrogen stream to remove non-adhered particles. Samples were sputter coated with Au/Pd for 20 minutes prior to visualization for an estimated coat depth of 15 nanometers. Microscope was operated under a working distance of 4.7 to 5.1 mm and an accelerating voltage of 5 to 15 kV.

Time of Flight Secondary Ion Mass Spectrometry (ToF-SIMs)

To analyze the distribution of each molecule within the powder, time-of-flight secondary ion mass spectrometry (ToF-SIMS) was performed on the powder of the combination pharmaceutical composition. ToF-SIMS depth profiles were acquired on an ToF.SIMS5 spectrometer (IONTOF, Muenster, Germany) using a 25 keV Bi3+ cluster ion source in the pulsed mode. Depth profiles were acquired in the non-interlaced mode using alternating analysis and sputter cycles. Data was acquired over a mass range of m/z=0 to 850 using a primary ion current of 0.035 pA in delayed extraction mode over a 100 micron×100 micron area centered within the sputter crater. Secondary ions of a given polarity were extracted and detected using a reflectron time-of-flight mass analyzer. The primary ion dose for each spectrum was 2.3×10¹¹ ion/cm². Sputtering was carried out using an gas cluster ion beam with 10 keV argon 1000 clusters rastered over a 500 micron×500 micron area for 7 seconds at a current of 7 nA giving a sputtering dose of 1.22×10¹⁴ ion/cm². Positive ion spectra were calibrated using the CH3+, C2H3+, and C3H5+ peaks. The negative ion spectra were calibrated using the CH—, OH—, and C2H— peaks. Calibration errors were kept below 20 ppm. Mass resolution (m/Δm) for a typical spectrum was 3400 for m/z=27 (pos) and 3600 for m/z=25 (neg).

Powder X-Ray Diffraction Analysis

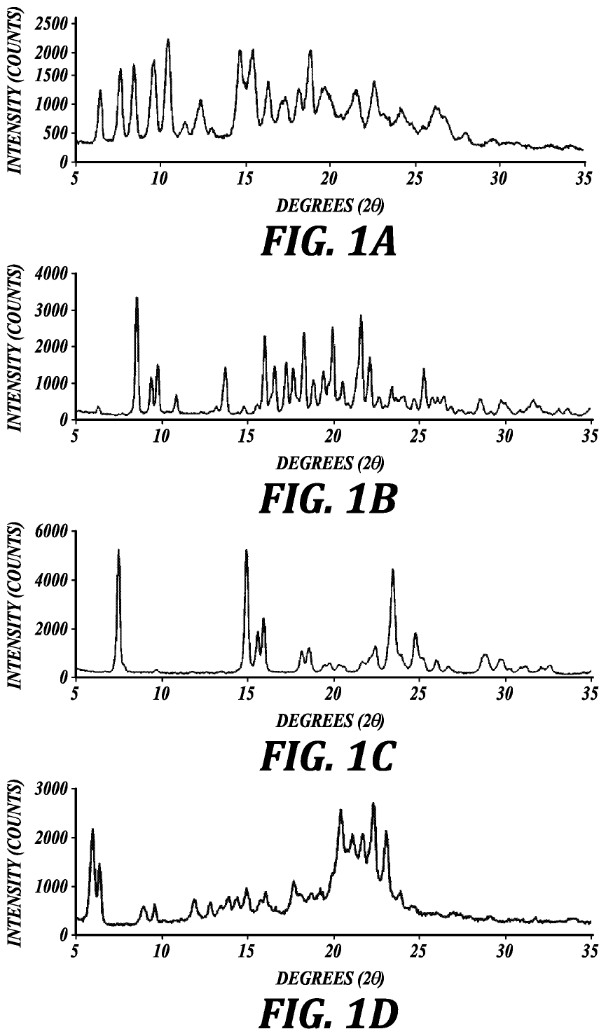

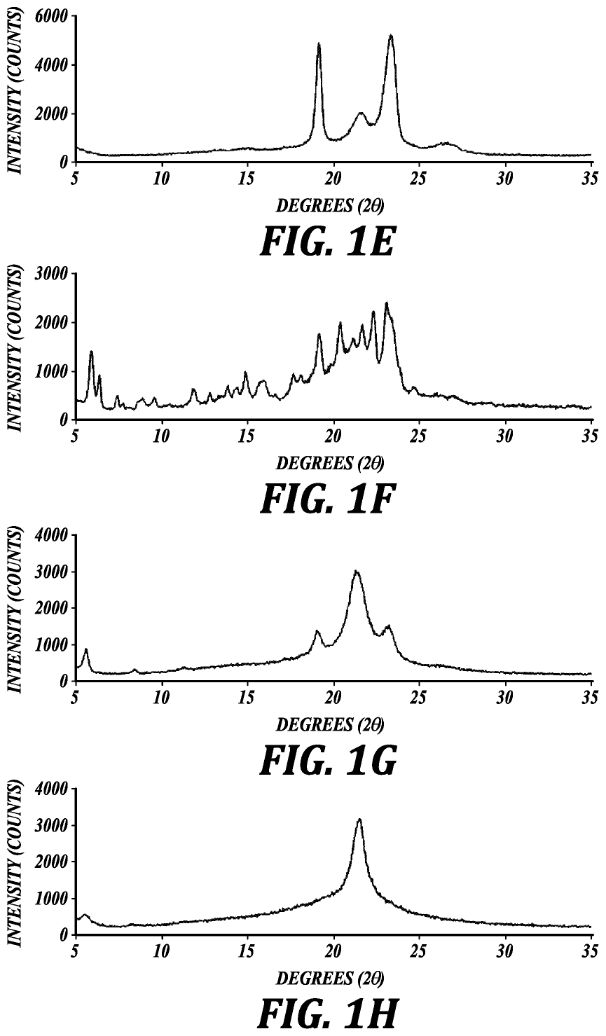

To determine the effects that controlled solvent removal may have on the physical structure of 5 components, LPV/RTV/TFV/DSPC and DSPE-PEG2000 together in a mixture, powder x-ray diffraction (PXRD) analysis was used to determine whether each molecule retained its original structure or assumed a new physical structure. The PXRD allowed evaluation of molecular spacing and crystallinity of the solid-state product. As shown in FIG. 1A-1H, individual constituents LPV/RTV/TFV/DSPC and DSPE-PEG2000 had respective diffraction patterns based on the crystal structure of each sample. As a result, diffraction patterns could provide a qualitative identification of crystalline materials. Analysis of the single crystal controls (FIGS. 1A through 1E) showed various peaks at the respective 2θ positions where Bragg's law was fulfilled. In Panel F, the diffraction pattern of the physical mixture of drugs and excipients was similar to that of DSPC alone due to the high mass % of this excipient in the formulation. However, additional peaks attributable to the other constituents were apparent particularly in the lower angle regions (5° to 15° 2θ). Relative to these diffraction patterns, the spray-dried drug-combination (with 2 lipid and lipid conjugate excipients) powder revealed two new diffraction peaks centered around 5.64° 2θ and 21.47° 2θ and none of the diffraction peaks from LPV/RTV/TFV and PEG-DSPE remained (FIG. 1H). These diffractions peaks were not attributable to any peak seen in the individual constituents. A further control batch was formulated using the same spray drying process but composed of lipid and lipid conjugate excipients alone and was then analyzed with PXRD (FIG. 1G). Interestingly, the absence of crystalline API produced additional peaks at the 19.1° 2θ and 23.1° 2θ positions. While overall diffraction patterns could provide a qualitative look at crystal identity, individual diffraction peaks could assign values to the spacing between molecular planes based on Bragg's law. In the spray-dried drug combination (and lipid and lipid conjugate excipient) powder, the diffraction peak at 5.64° 2θ and 21.47° 2θ corresponded to d-spacing of 15.66 Å and 4.14 Å, respectively. The peaks at 5.64° 2θ and 21.47° 2θ indicated the presence of long-range order. In the drug-free control, primary diffraction peaks were observed at 5.6° 2θ and 21.3° 2θ with secondary peaks at 19.1° 2θ and 23.1° 2θ (FIG. 1G). These secondary peaks were believed to be the result of PEG re-crystallization based on the known 2θ positions of PEG and prevalence of PEG residues in the formulation (˜20% w/w). With the inclusion of either hydrophilic drug (TFV) or hydrophobic drugs (LPV/RTV) these secondary peaks were absent. Collectively, these data indicated that the solvent removal process allowed for the individual constituents to arrange in an organized pattern unique from the drug-free and free drug controls. The loss of diffraction peaks with the inclusion of crystalline drugs could be due to a regional dilution effect on the concentration of PEG thus preventing phase separation. Alternatively, this is indicative of interactions between drug and PEG that prevent inter- and intra-polymeric ordering of PEG residues.

Differential Scanning Calorimetry for Confirmation of New Physical Structure

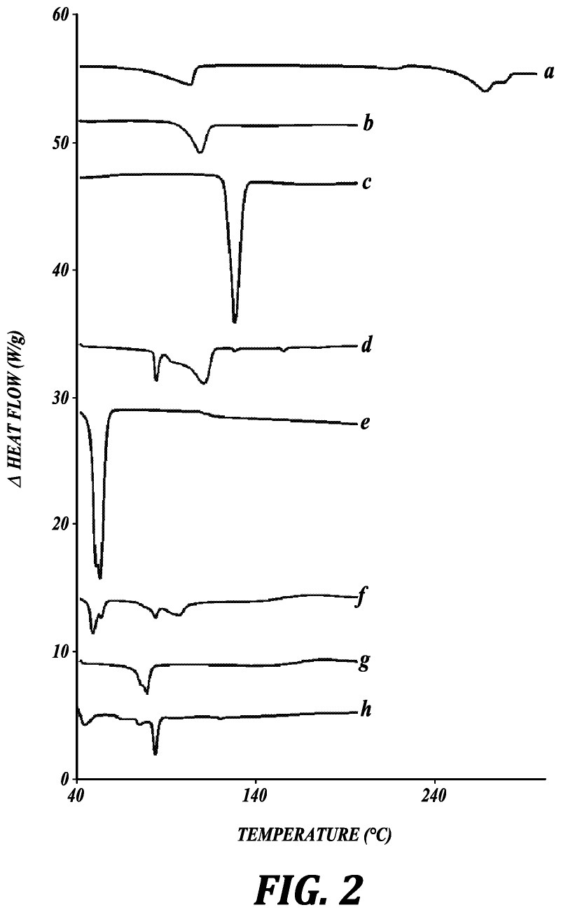

To further understand the physical structure of our spray dried combination, differential scanning calorimetry was employed as a complementary technique to PXRD. In the spray dried formulation, a single endothermic transition could be seen with an onset temperature of 70.28° C. and a melting point at 74.29° C. (FIG. 2, line G). This endotherm occurred at a position independent of individual drug and excipient controls, indicating that it was not the melting of unchanged crystalline drug or excipient. The presence of this endotherm indicated that there was some degree of structure in the spray dried powder, and the breaking of the nonbonding interactions in this structure was detectable through DSC. Unfortunately, the thermogram also contained a broad exotherm beginning at temperatures >120° C. and extending until the end of the heating ramp. A possible source of this exotherm was the mass loss from heating of the drug combination powder formulation, which was observed to be ˜3.5% based on TGA measurements at a ramp rate of 10° C./minute to 200° C. The weight change of the combination drug powder was likely due to bound water adsorbed to the powder, which was characterized via Karl Fisher titrations to be ˜5-8% by mass (data not shown), but could also be the result of degradation. The thermal characterization of the spray dried powder supported the presence of long range order that breaks down as a function of temperature.

Time of Flight Secondary Ion Mass Spectrometry (Tof-SIMs) and SEM Analysis

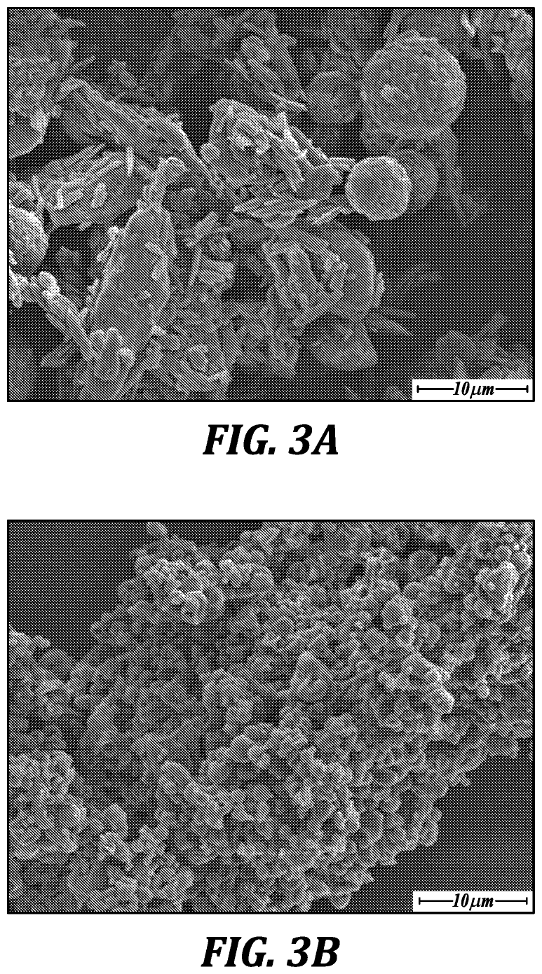

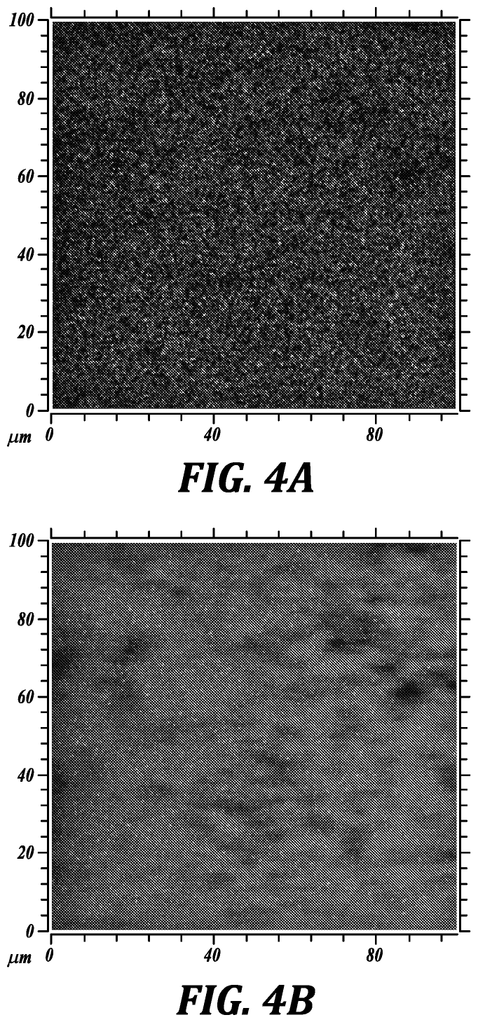

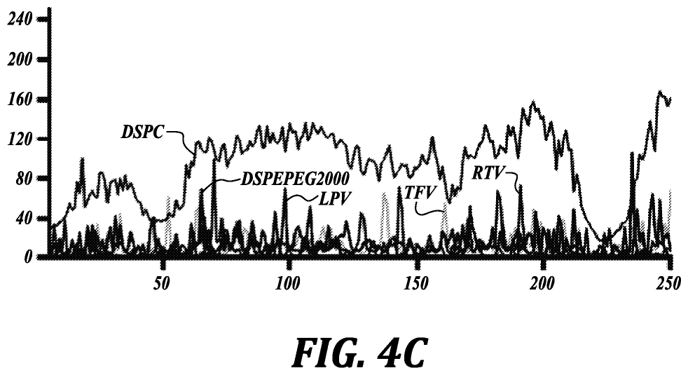

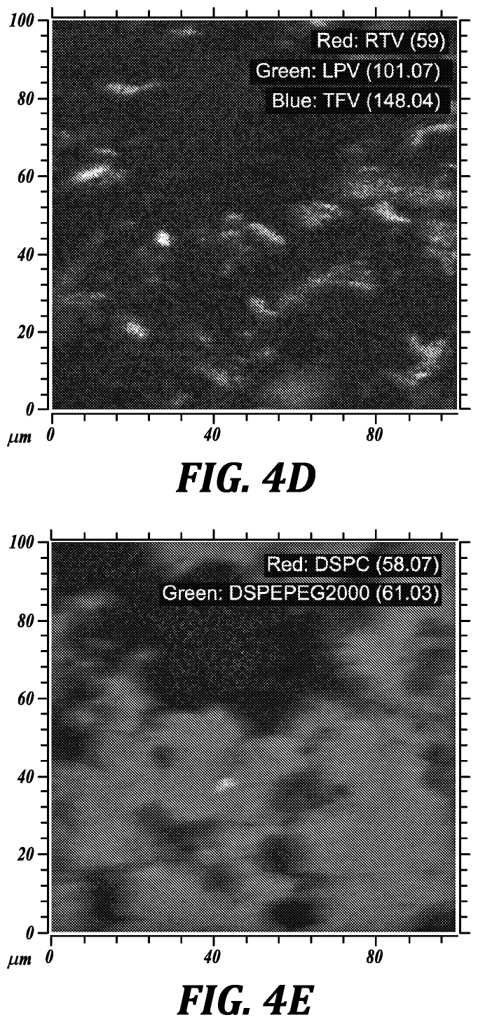

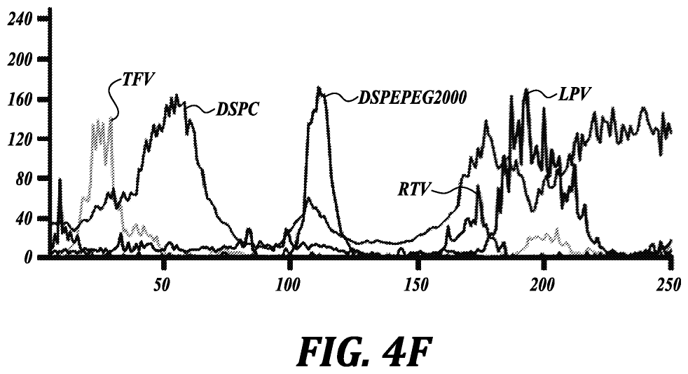

To understand the homogeneity and molecular distribution of the three drugs and two lipid and lipid conjugate excipients in the combination pharmaceutical composition powder, ToF SIMs and SEM techniques were utilized. ToF SIMs is a surface analysis technique that can provide information on the molecular surface structure of a solid material. By tuning specific fragments to the individual constituents of the combination pharmaceutical composition, ToF SIMs could be used to map the distribution of drugs and excipients in a solid powder. SEM allowed for the visualization of individual particles in the sub-micron scale and could provide valuable information on particle morphology and homogeneity. FIGS. 3A and 3B showed the change in morphology associated with the spray drying process (FIG. 3B) relative to a physically mixed control (FIG. 3A). The morphology of the spray dried material did not retain any of the physical characteristics associated with the individual constituents but rather had a homogeneous, spherical shape (˜1 to 5 μM) associated with the atomized droplets of feedstock solution. Further ToF-SIMs analysis (FIGS. 4A-4C) of the spherical particles observed in SEM revealed that the drugs and excipients were very well distributed. The control physical mixture did not provide homogeneous drugs or lipid and lipid conjugate excipients distribution (FIGS. 4D-4F). These data indicated that there was no preferential accumulation of API or excipients within a single particle and each individual particle had a uniform composition and shape. The ToF-SIMs analysis provided sufficient resolution to distinguish individual drug crystals in the physical mixture. The current techniques have shown the effects of spray drying on particle morphology, homogeneity and molecular distribution. Rapid removal of feedstock solvent from atomized droplets produced homogeneous particles with a uniform distribution of three very physicochemically distinct compounds with lipid and lipid conjugate excipients. Taken together with the PXRD and DSC experiments, these data indicated that the interactions between drug and excipients was facilitated through controlled solvent removal to form new structural conformations that occurred on a submicron scale. In addition, the structure observed in spray-dried material was not attained through physical mixture.

Multi-Drug Motif (MDM) Formation by Controlled Solvent Evaporation Process is Applicable for a Number of Drug Combination

To understand whether the MDM structure formation using the process of the present example could extend to other drug combination, additional drug combinations were evaluated. These combinations included the following: hydrophobic lopinavir and ritonavir in the drug combination above were replaced with dolutegravir, rilpivirine, or both. Hydrophilic tenofovir either replaced or added in combination with lamivudine or emtricitabine.

The new drug combinations also formed the MDM structure using the composition and process described for LPV/RTV/TFV with two lipid and lipid conjugate excipients. These results were summarized in Table 1. As PXRD was a good indicator of MDM formation, it was used to assess the structural features of MDM composition powder. Altering the drug composition listed in Table 1 still produced the MDM characteristics similar to that of the LPV/RTV/TFV combination. Collectively, these data indicate that the controlled solvent removal enabled the formation of a number of repeating multi drug motifs within each combination.

TABLE 1
Demonstration of different drug compositions successful
in producing ordered multi-drug-combination structures.

| | | | | | Formation of Multi- |
|---|---|---|---|---|---|
| | Hydrophilic Drugs | | Hydrophobic Drugs | | Drug Motif (MDM) |
| | (Log P < 1) | | (Log P > 2) | | By XRD |
| Lead | — | Tenofovir | Lopinavir | Ritonavir | Yes¹ |
| Combination | | | | | |
| Back up | Lamivudine | Tenofovir | Lopinavir | Ritonavir | Yes |
| Regimens | Lamivudine | Tenofovir | Dolutegravir | — | Yes |
| | Lamivudine | Tenofovir | Dolutegravir | Rilpivirine | Yes |
| | — | — | Lopinavir | Ritonavir | Yes |
| | — | Tenofovir | — | — | Yes |
| | — | — | Lopinavir | — | No |

¹Representative XRD pattern for this combination is presented in FIG. 1H.

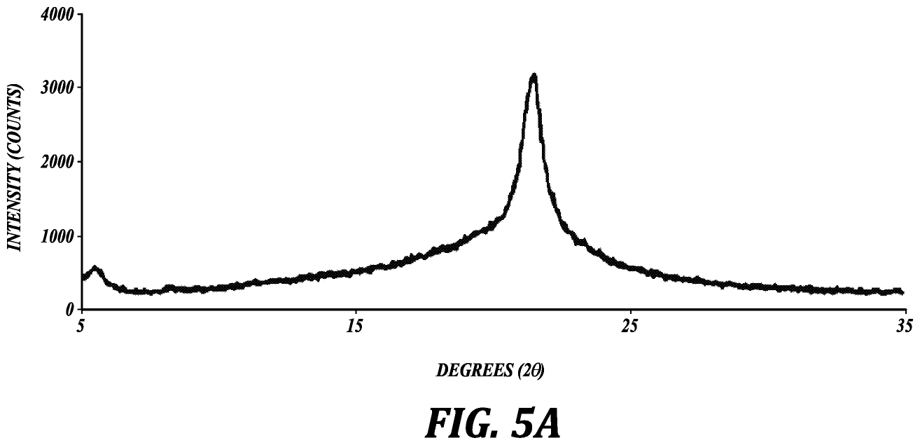

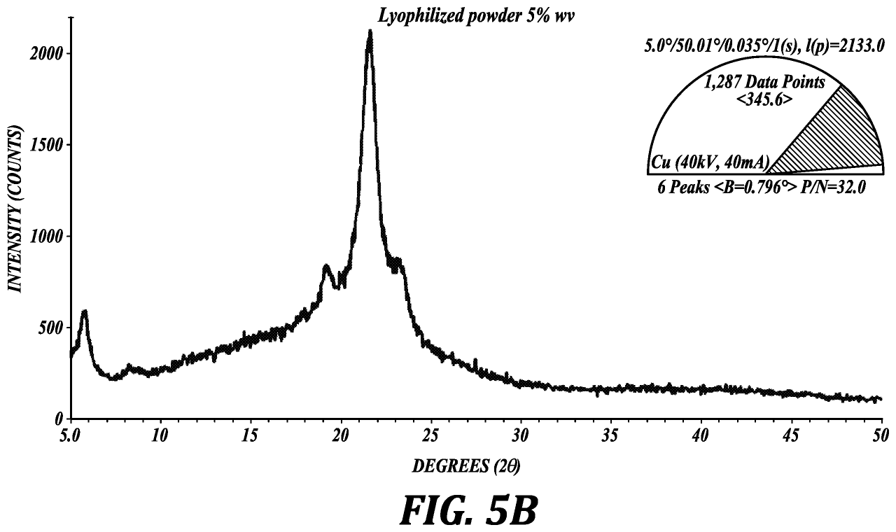

With respect to uncontrolled process of solvent removal, studies using rotary evaporation techniques (which was also used in manufacture of certain pharmaceutical liposome preparations) were carried out. As shown in Table 2, with the same therapeutic agents and excipients, rotary evaporation method did not yield MDM structure in a consistent manner compared to controlled solvent removal using the spray-drying process described above. In addition, whether solvent removal of the same set of drugs and lipid and lipid conjugate excipients in the same composition by freeze-drying process could produce MDM structure in the powder product was also investigated. The freeze-drying process was not able to produce MDM process as verified by X-ray (PXRD) analysis (FIGS. 5A and 5B).

TABLE 2
Various controlled solvent removal methods and success
in producing ordered multi-drug combination structures.

| | | | | | Formation of Multi- |
|---|---|---|---|---|---|
| | Hydrophilic Drugs | | Hydrophobic Drugs | | Drug Motif (MDM) |
| | (Log P < 1) | | (Log P > 2) | | By XRD |
| Rotary | — | Tenofovir | Lopinavir | Ritonavir | Variable |
| Evaporation | — | — | Lopinavir | Ritonavir | No |
| | — | Tenofovir | — | — | No |
| | — | — | Darunavir | — | No |
| Spray | — | Tenofovir | Lopinavir | Ritonavir | Yes¹ |
| Drying | Lamivudine | Tenofovir | Dolutegravir | — | Yes |
| | Lamivudine | Tenofovir | Dolutegravir | Rilpivirine | Yes |
| | — | — | Lopinavir | Ritonavir | Yes |
| | — | Tenofovir | — | — | Yes |

¹Representative XRD pattern for this combination is presented in FIG. 1H.

A range of lipid/lipid conjugate and drug composition were investigated, and the described composition (DSPC:DSPE-PEG₂₀₀₀:LPV:RTV:TFV in a ratio of 103.5/11.5/12/3/15) was found to be optimal (Table 3). The data indicate that the total drug to lipid ratio can be increased by about 5 fold that of the lead composition and still produce MDM powder structure.

TABLE 3
Variation of Drug to lipid loading ratios in
LPV/RTV/TFV Lead Formulation.

| | Formation of Multi-Drug |
|---|---|
| DSPC:DSPE-PEG₂₀₀₀:LPV:RTV:TFV | Motif (MDM) By XRD |
| 103.5/11.5/12/3/15 (Lead Ratio) | Yes/No (Variable) |
| 103.5/11.5/24/6/30 | No |
| 103.5/11.5/48/12/60 | No |
| 103.5/11.5/60/15/75 | No |
| 103.5/11.5/12/3/15 | No |
| 103.5/11.5/12/3/30 | No |
| 103.5/11.5/12/3/60 | No |
| 103.5/11.5/12/3/75 | No |
| 103.5/11.5/12/3/5 | No |
| 103.5/11.5/96/24/15 | No |

To address the question of whether MDM formation was limited a specific spray-drying instrument, two spray-dryers were evaluated: one from ProCept and the other from Buchi. While the two spray dryers had different configuration and requirements for operation, both were able to provide controlled solvent removal process necessary to provide a product with MDM motifs in the combination pharmaceutical composition powder.

TABLE 4
Instrumental variation in controlled solvent removal.

| | | | | | Formation of Multi- |
|---|---|---|---|---|---|
| | Hydrophilic Drugs | | Hydrophobic Drugs | | Drug Motif (MDM) |
| | (Log P < 1) | | (Log P > 2) | | By XRD |
| Buchi | — | Tenofovir | Lopinavir | Ritonavir | Variable |
| Rotary | | | | | (Yes/No) |
| Evaporator | | | | | |
| ProCept | — | Tenofovir | Lopinavir | Ritonavir | Yes¹ |
| | Lamivudine | Tenofovir | Dolutegravir | — | Yes |
| | Lamivudine | Tenofovir | Dolutegravir | Rilpivirine | Yes |
| | — | Tenofovir | — | — | Yes |
| Buchi Spray | — | Tenofovir | Lopinavir | Ritonavir | Yes |
| Dryer | — | Tenofovir | — | — | Yes |
| | — | — | Lopinavir | — | Yes |
| | — | — | — | Ritonavir | Yes |

¹Representative XRD pattern for this combination is presented in FIG. 1H.

Thus, the present Example describes methods for controlled solvent removal from a fully solubilized mixture of 3 API and 2 excipients by spray-drying, which lead to formation of novel multi-drug motifs in the powder form. These motifs were verified as unified structures by powder x-ray diffraction. XPRD analysis of spray dried powders revealed that the final MDM product is not completely amorphous and contains long-range order distinct from the individual constituents. This long-range order can increase stability of the drug combination powder product relative to amorphous materials. Differential scanning calorimetry analysis also revealed that after undergoing a controlled solvent removal process, the newly formed powder underwent a single endothermic transition at 74.29° C. distinct from any of the individual constituents alone. The collective transition at a single distinct temperature supported the MDM structure. Morphological analysis with SEM shows a homogeneous morphology from controlled solvent removal distinct from the physical mixture of the same components. Further surface analysis by ToF-SIMs also showed that the combination pharmaceutical composition powder had greater homogeneity and molecular distribution of the materials (FIG. 4). Collectively, these data indicated that controlled solvent removal from hydrophobic and hydrophilic drugs and excipients under described conditions form novel MDM structures.

The combination pharmaceutical composition powder exhibited two diffraction peaks at 5.64° 2θ and 21.47° 2θ, corresponding to d-spacing of 15.66 Å and 4.14 Å, respectively (FIG. 1H). These two molecular planes (d-spacing) can be attributed to: (1) the behavior of the phospholipidic excipients in solution prior to evaporation and (2) the rate of feedstock evaporation associated with spray drying. The data indicated that the combination pharmaceutical composition powder had structural features similar to hydrated DSPC even in the presence of 3 API and pegylated DSPE. In contrast, multidrug combinations composed of hydrophobic ritonavir, etravirine and efavirenz were previously produced as amorphous solid dispersions. The data showed a physical transformation from the pure crystalline forms of the therapeutic agents, but not complete amorphous conversion. Instead, the combination pharmaceutical composition retains many of the macroscopic properties associated with lipid and lipid conjugate excipients (diffraction at 5.6° 2θ and 21.3° 2θ) in conjunction with well dispersed therapeutic agents within those excipients. These features provide a great advantage for combination drug delivery and for improving therapeutic effects of the therapeutic agents.

Thus, the present Example demonstrates that controlled solvent removal allowed for the ordering of lipid and lipid conjugate excipients. In addition, the data show that within the ordering of lipid and lipid conjugate excipients there are nonbonding interactions between drugs and excipients on a submicron scale that was not achieved with the physical mixture of these components. These nonbonding, stable interactions can facilitate the formation of supramolecular structures in aqueous solution. The structures do not form bilayers but produce long acting behavior for both hydrophilic and hydrophobic drug over two weeks in non-human primates. These novel structures are different from less stable liposome bilayers and can explain the unique and prolonged bioperformance. Furthermore, spray drying was demonstrated as a scalable and reproducible method for MDM formation.

Characterization of Suspension of MDM Combination Pharmaceutical Composition

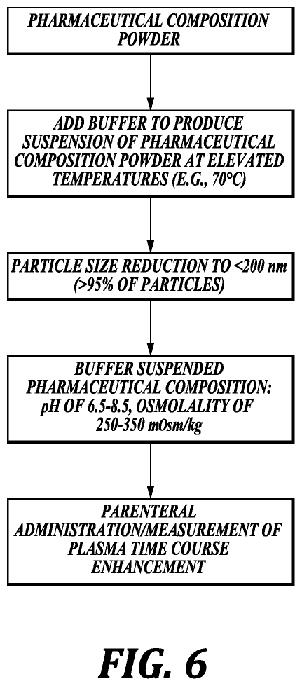

FIG. 6 shows a flow chart schematic for suspension of the combination pharmaceutical composition having MDM structure. Referring to FIG. 6, a MDM combination pharmaceutical composition (“MDM composition”) of the present example is suspended in an aqueous buffer at 70° C., followed by particle size reduction to less than 200 nm (for greater than 95% of the particles). The suspended MDM combination pharmaceutical composition can have a pH between 6.5 to 8.5 and an osmolality of from 250 to 350 mosm/kg. The suspended MDM composition can then be used in parenteral administration or further studies.

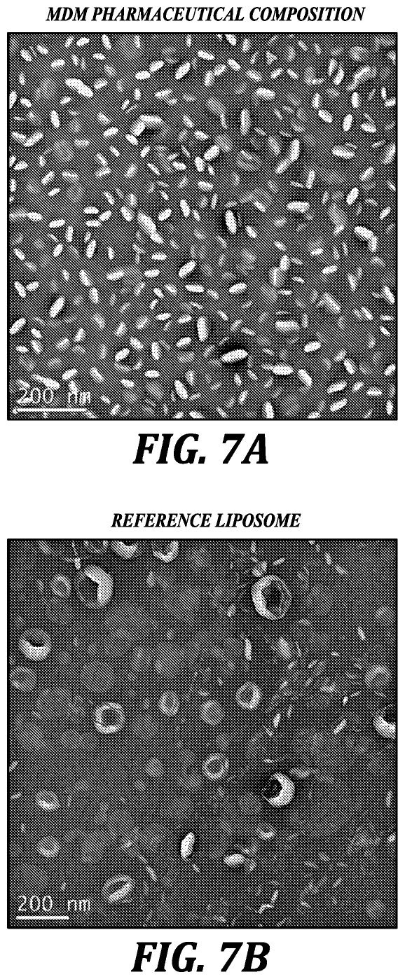

To understand the structure of suspended MDM composition compared to other lipid-based suspensions, transmission electron microscopy (TEM) was carried out. The MDM composition was suspended in aqueous buffer and homogenized for particle size reduction to form suspended MDM composition nanoparticles. Referring to FIG. 7A, the nanoparticles were visualized by TEM. A comparative lipid-based formulation was made by suspending lipid/lipid conjugate excipients and using extrusion to form liposomes. These liposomes were also characterized by TEM (FIG. 7B).

When suspended, the MDM composition has a different structure from self-assembled reference liposomes. The elongated drug/lipid complex of the MDM composition does not show a bilayer structure.

To understand the structure of the MDM powder relative to other products, powder X-ray diffraction (PXRD) was carried out. Lopinavir, ritonavir and tenofovir were completely dissolved in chloroform/water/ethanol without lipid and lipid conjugate excipients. The solution was placed in a rotary evaporator at high pressure, rotation speed and temperature to produce rapid solvent removal and amorphization of the material. Amorphous conversion was confirmed with PXRD. The commercially stable Kaletra formulation of lopinavir and ritonavir was also analyzed with PXRD to confirm amorphous structure. Acquisition of various samples were performed on two independent instruments and signals were normalized for comparison.

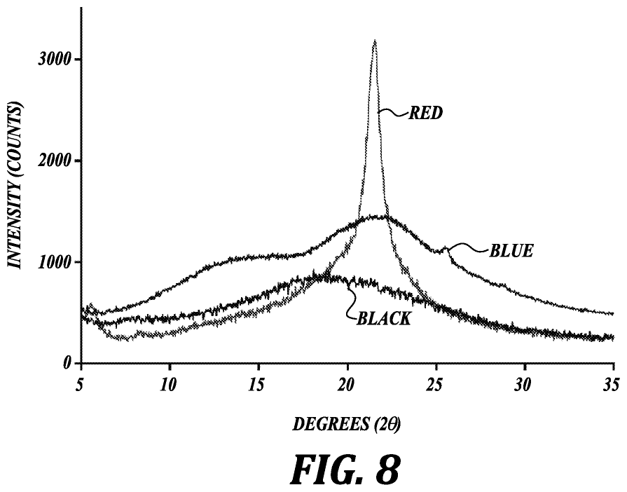

Referring to FIG. 8, the MDM composition formed through controlled solvent removal showed characteristic MDM structure (red). In contrast, a mixture of LPV/RTV/TFV that has undergone uncontrolled solvent removal can convert completely to amorphous material as demonstrated by the characteristic “halo” in the diffraction (black). A crushed comparator product, Kaletra (LPV/RTV), was also analyzed and produces a similar amorphous pattern (blue).

Thus, LPV/RTV/TFV undergoing rapid, uncontrolled solvent removal becomes fully amorphous in the absence of lipid and lipid conjugate excipients. Crushed tablets of commercially available Kaletra (LPV/RTV) were also amorphous even in the presence of film coat excipients. When undergoing a controlled solvent removal process with lipid/lipid conjugate excipients, LPV/RTV/TFV formed a structure that was clearly different from amorphous powder.

### Example 2. Suspended Product Exhibiting Long-Acting Plasma Pharmacokinetics of Antiviral Drugs

The experiment was carried out to determine if a single subcutaneous dose of suspended MDM combination pharmaceutical composition powder (“MDM composition”) can enable long acting plasma circulation of three combination drugs in non-human primates.

Four primates (Macaca nemestrina) were administered with a suspension of the MDM composition prepared as described in Example 1 and free drug in a cross-over study at a normalized dose of 25 mg/kg lopinavir (LPV), 14.3 mg/kg ritonavir (RTV) (2:1 mole to mole), and 17.1 mg/kg tenofovir (TFV) subcutaneously. Free formulation of LPV, RTV, and TFV was prepared in 20 mM NaHCO₃-buffered water (pH 7.4) with 0.7% NaCl, 8% DMSO, and 0.1% Tween20 and had the same final drug concentrations as the suspended MDM composition.

TABLE 5
Utility of MDM composition to produce suspended product that exhibits
long-acting plasma pharmacokinetics of antiviral drugs.

| | [Lopinavir]ng/ml | | | [Ritonavir]ng/ml | | | [Tenofovir]ng/ml | | |
|---|---|---|---|---|---|---|---|---|---|
| | | MDM | | | MDM | | | MDM | |
| | | compo- | | | compo- | | | compo- | |
| Time (hr) | Free | sition | Ratio^{b} | Free | sition | Ratio^{b} | Free | sition | Ratio^{b} |
| 0 | 0 | 0 | 1 | 0 | 0 | 1 | 0 | 0 | 1 |
| 0.5 | 670 | 280 | 0.4 | 960 | 410 | 0.4 | 5010 | 1290 | 0.3 |
| 1 | 1930 | 1850 | 1 | 2920 | 2570 | 0.9 | 3550 | 1290 | 0.4 |
| 3 | 910 | 2390 | 2.6 | 2850 | 3050 | 1.1 | 2570 | 1190 | 0.5 |
| 5 | 320 | 2590 | 8.1 | 900 | 2200 | 2.4 | 270 | 1240 | 4.6 |
| 8 | 700 | 4230 | 6 | 740 | 4570 | 6.2 | 10 | 590 | 59 |
| 24 | 160 | 3260 | 20.4 | 0 | 4090 | >409.0 | 10 | 700 | 70 |
| 48 | 10 | 30 | 3 | 0 | 0 | 1 | 0 | 2270 | >227.0 |
| 120 | NA | 20 | NA | NA | 1370 | NA | NA | 640 | NA |
| 168 | 0 | 80 | >8.0 | 0 | 1320 | >132.0 | 0 | 250 | >25.0 |

Pharmacokinetic Analysis

| | | | | | | | | | |
|---|---|---|---|---|---|---|---|---|---|
| AUC^{a} | 3.83 | 69.6 | 18.2 | 1.39 | 19.4 | 14.0 | 56.6 | 395 | 7.0 |
| (μg * h/mL) | | | | | | | | | |
| T_{1/2}^{c}, | 6.5 | 27.7 | 4.3 | 2.7 | 86.6 | 31.8 | 0.6 | 38.5 | 61.6 |
| apparent | | | | | | | | | |
| (hr) | | | | | | | | | |

^{a}Area under the curve (AUC) was calculated from plasma drug concentrations using the trapezoidal rule, over 168 hours.
^{b}Ratio are presented as MDM composition/free drug.
^{c}Apparent terminal half-life is calculated using the final points in the concentration time curve of LPV, RTV, and TFV. Additional sampling past 1 week may affect this value.
NA—Not available

When administered the same dose of MDM composition as free drug, the MDM composition produced persistently higher plasma concentrations of all three combination drugs after 5 hours. Subsequent pharmacokinetic analysis showed that overall exposure was also increased significantly when administered as a MDM composition. The terminal half-life of all three drugs were also increased when administered as a MDM composition.

### Example 3. MDM Composition in Suspension to Enable Extension of Antiviral Plasma Circulation to Two Weeks

This experiment was conducted to determine if the enhanced plasma circulation could be extended to two weeks with further sampling and determine whether the enhanced plasma circulation of three drugs is reproducible, a follow up study with the same protocol described in Example 2 was conducted. A new batch of MDM composition powder was formulated with a slightly different composition (4:1 LPV/RTV versus 2:1 LPV/RTV) and a pharmacokinetic study was performed.

A suspended MDM composition prepared according to Example 1 was administered at a dose of 25 mg/kg of lopinavir, 7 mg/kg of ritonavir (4:1 mole to mole) and 10.6 mg/kg of tenofovir. Free formulation of LPV, RTV, and TFV was prepared in 20 mM NaHCO₃-buffered water (pH 7.4) with 0.7% NaCl, 8% DMSO, and 0.1% Tween20 and had the same final drug concentrations as the suspended MDM composition.

TABLE 6
Utility of MDM composition in suspension to enable extension of
antiviral plasma circulation to two weeks.

| | | Lopinavir | | | Ritonavir | | | Tenofovir | | |
|---|---|---|---|---|---|---|---|---|---|---|
| | | | MDM | | | MDM | | | MDM | |
| | | | compo- | | | compo- | | | compo- | |
| Parameter | Units | Free | sition | Ratio | Free | sition | Ratio | Free | sition | Ratio |
| AUC₀ to t^{a} | μg * h/mL | 4.35 | 10.98 | 2.52 | 1.37 | 4.8 | 3.50 | 14.4 | 416.55 | 28.93 |
| T_{1/2, terminal} | hr | 8.65 | 476.94 | 44.06 | 7.99 | 44.06 | 5.51 | 8.01 | 65.33 | 8.16 |

^{a}AUC0-24h for Free, AUC0-336h.
AUC = area under the plasma drug concentration-time curve
t1/2 = apparent terminal plasma drug half-life

Although the composition of MDM composition changed slightly, there was a continued enhancement in exposure when compared to freely solubilized drug. This effect was also seen in the apparent terminal half-life of all three drugs. The MDM composition could enable the transformation of short acting antiviral injections to long acting injections.

### Example 4. Comparison of Conventional Dosage Form of LPV/RTV Taken Orally in Humans Compared to Orally in Primates

| | Non-Human Primates | Human |
|---|---|---|
| | Oral administration | Oral administration |
| | LPV/RTV (1:1) | LPV/RTV (4:1) |
| AUC_{μg*hr*kg/(mL*mg)}, | 1.47 | 10.45 |
| LPV | | |
| T_{1/2}, ₐₚₚ₍ₕᵣ₎ | — | 4-6 hours |

AUC (area under the curve, or total drug exposure) of the active drug lopinavir was dose normalized across species and route of administration by dividing the total exposure by the dose administered (μg*hr*kg/(mL*mg)). Dashes represent unreported data. Apparent half-life is reported in hours.

In commercially available formulations of Kaletra (lopinavir/ritonavir), the half-life of the active drug lopinavir is 4-6 hours which requires twice a day dosing in humans. In non-human primates, the total AUC of lopinavir (PO) is 10-fold lower than humans even in the presence of more RTV (metabolic inhibitor).

MDM compositions in suspension could enable an injectable, long acting form of LPV/RTV with more overall lopinavir exposure (2.5×) and longer half-life (44×) than freely solubilized drug (see Example 3).

### Example 5. Comparison of Conventional Dosage for TFV Given Intravenously (IV) in Humans Compared to Subcutaneously (SC) in Primates

| | Non-human Primates | Human |
|---|---|---|
| | SC | IV |
| AUC_{μg*hr*kg/(mL*mg)} | 2.1 | 5.6 |
| T_{1/2, app(hr)} | 8 | 6.6 |

AUC (area under the curve, or total drug exposure) of the active drug lopinavir was dose normalized across species and route of administration by dividing the total exposure by the dose administered (μg*hr*kg/(mL*mg)). Apparent half-life is reported in hours.

Tenofovir is only commercially available in prodrug form (TDF or TAF) and is dosed daily. IV administration of active TFV has a half-life of 6.6 hours in humans and available SC data in non-human primates shows an 8 hour half-life in non-human primates. MDM compositions in suspension can enable an injectable, long acting form of active TFV without needing prodrug formulation with a 8-fold increase in half-life and 28.9 fold increase in exposure compared to freely solubilized drug (see Example 3).

While illustrative embodiments have been illustrated and described, it will be appreciated that various changes can be made therein without departing from the spirit and scope of the invention.

1. A pharmaceutical composition, comprising:
a hydrophobic therapeutic agent having a log P value of 1 or greater;
a hydrophilic therapeutic agent having a log P value of less than 1; and
one or more compatibilizers comprising a lipid excipient, a lipid conjugate excipient, or a combination thereof;
wherein the pharmaceutical composition is a solid, and
wherein the pharmaceutical composition has a powder X-ray diffraction pattern comprising at least one peak having a signal to noise ratio of greater than 3, wherein the peak is different from the diffraction peaks of each individual component of the pharmaceutical composition.

2. The pharmaceutical composition of claim 1, comprising a unified repetitive multi-drug motif structure and/or a long range order in the form of a repeating pattern.

3-6. (canceled)

7. The pharmaceutical composition of claim 1, wherein the pharmaceutical composition is not amorphous; wherein the pharmaceutical composition exhibits a phase transition temperature different from the transition temperature of each individual component when assessed by differential scanning calorimetry; and/or wherein the pharmaceutical composition is in the form of homogeneous distribution of each individual therapeutic agent when viewed by scanning electron microscopy.

8-10. (canceled)

11. The pharmaceutical composition of claim 1, wherein the pharmaceutical composition comprises the hydrophobic therapeutic agent and the hydrophilic therapeutic agent each independently in an amount of 2 wt % or more and 20 wt % or less; wherein the hydrophobic therapeutic agent comprises a hydrophobic antiviral agent, a hydrophobic anti-infective agent, or a combination thereof, and wherein the hydrophilic agent comprises an antiviral agent, an anti-infective agent, or a combination thereof.

12. The pharmaceutical composition of claim 11, wherein the hydrophobic antiviral agent is selected from lopinavir, ritonavir, dolutegravir, rilpivirine, atazanavir, dorunavir, efevirenz, and raltigravir; and wherein the hydrophilic antiviral agent is selected from tenofovir, lamivudine, abacavir, tenofovir disoproxil fumarate, tenofovir alafenamide, and emtricitabine.

13-15. (canceled)

16. The pharmaceutical composition of claim 1, wherein the pharmaceutical composition comprises the one or more compatibilizers in an amount of 60 wt % or more and 95 wt % or less, and wherein the one or more compatibilizers comprise at least one lipid excipient in an amount of 50 wt % or more and 80 wt % or less and at least one lipid conjugate excipient in an amount of 19 wt % or more and 25 wt % or less.

17-19. (canceled)

20. The pharmaceutical composition of claim 1, wherein the lipid excipient is selected from 1,2-distearoyl-sn-glycero-3-phosphocholine (DSPC), 1,2-dimyristoyl-sn-glycero-3-phosphocholine (DMPC), and 1,2-dipalmitoyl-sn-glycero-3-phosphocholine (DPPC); and wherein the lipid conjugate excipient comprises a covalent conjugate of a lipid with a hydrophilic moiety.

21-22. (canceled)

23. The pharmaceutical composition of claim 1, comprising a molar ratio of hydrophobic therapeutic agent and hydrophilic therapeutic agent to the one or more compatibilizers of from 30:115 to 71:40.

24-25. (canceled)

26. A suspension comprising the pharmaceutical composition of claim 1, wherein the pharmaceutical composition is dispersed in an aqueous solvent in the form of a suspension.

27. A method of making a pharmaceutical composition, comprising:
dissolving a hydrophobic therapeutic agent having a log P value of 1 or greater; a hydrophilic therapeutic agent having a log P value of less than 1; and one or more compatibilizers comprising a lipid excipient, a lipid conjugate excipient, or a combination thereof in an alcoholic solvent at a temperature of 65 to 75° C. to provide a solution,
maintaining the solution at a temperature of 65 to 75° C.;
spraying the solution from an inlet nozzle and evaporating the alcoholic solvent in a chamber to provide the pharmaceutical composition comprising the hydrophobic therapeutic agent, the hydrophilic therapeutic agent, and the one or more compatibilizers in the form of a powder.

28. (canceled)

29. The method of claim 27, wherein the alcoholic solvent comprises methanol, ethanol, propanol, or any combination thereof.

30. The method of claim 27, wherein the alcoholic solvent further comprises water.

31. (canceled)

33. The method of claim 27, wherein the alcoholic solvent is at a temperature of 50° C. to 65° C. or more.

34-35. (canceled)

36. The method of claim 27, wherein spraying from an inlet nozzle comprises maintaining an inlet temperature of 65° C. to 75° C.

37. The method of claim 27, wherein the chamber is maintained at a pressure of 20 mBar to 30 mBar.

38-40. (canceled)

41. A method of administering the pharmaceutical composition of claim 1, comprising:
mixing the pharmaceutical composition of claim 1 with an aqueous solvent to provide an aqueous dispersion comprising the pharmaceutical composition; and
parenterally administering the aqueous dispersion to a subject.

42. The method of claim 41, wherein the aqueous dispersion comprises a supramolecular organization of the hydrophobic therapeutic agent, the hydrophilic therapeutic agent, and the one or more compatibilizer; and wherein the aqueous dispersion comprising the pharmaceutical composition does not comprise a lipid layer excipient, a lipid bilayer excipient, a liposome, or a micelle.

43-48. (canceled)

49. The method of claim 41, wherein the aqueous dispersion is parenterally administered once every 7 to 28 days.

50. The method of claim 41, wherein the aqueous dispersion comprising the pharmaceutical composition provides 2 to 20 fold higher exposure of each individual therapeutic agent in non-human primates, when administered subcutaneously.

51. The pharmaceutical composition of claim 41, wherein the aqueous dispersion comprising the pharmaceutical composition has a half-life greater than the half-life of each freely solubilized individual therapeutic agent.
